(19) United States
(12) Patent Application Publication
(10) Pub. No.: US 2008/0124717 A1
(43) Pub. Date: May 29, 2008

(54) Method and apparatus for label-free electronic real-time double-stranded nucleic acid detection

(76) Inventors: Scott Manalis, Cambridge, MA (US); Johnson Hou, Taipei (TW)

Correspondence Address: CHOATE, HALL & STEWART LLP, TWO INTERNATIONAL PLACE, BOSTON, MA, 02110, US

(21) Appl. No.: 11/634,501

(22) Filed: Dec. 6, 2006

Related U.S. Application Data
(63) Continuation-in-part of application No. 10/201,333, filed on Jul. 23, 2002.
(60) Provisional application No. 60/749,742, filed on Dec. 13, 2005.
(60) Provisional application No. 60/329,204, filed on Oct. 12, 2001.

(51) Int. Cl. C12Q 1/68; C12M 1/34

(52) U.S. Cl. 435/6; 435/287.2

(57) ABSTRACT

In various embodiments, the present invention is a method and apparatus for label-free detection and optional quantification of double-stranded nucleic acid comprising binding a first layer comprising a charged species to a sensing surface having an associated first charge, wherein the first layer confers to the sensing surface a second charge or a neutral charge on a net basis, performing at least one cycle of DNA amplification to produce a double-stranded nucleic acid, and measuring a property of the interaction between the first layer and the double-stranded nucleic acid after the at least one cycle of DNA amplification. The present invention may be used in a cyclic manner, corresponding with the cyclic nature of DNA amplification processes.

### CROSS-REFERENCE TO RELATED APPLICATIONS/PRIORITY CLAIM

This application claims priority to Provisional Patent Application No. 60/749,742, filed on Dec. 13, 2005, and is a continuation-in-part of U.S. patent application Ser. No. 10/201,333, filed on Jul. 23, 2002, which claims priority under 35 U.S.C. § 119(e) to U.S. provisional patent application, 60/329,204, filed Oct. 12, 2001, all of which are incorporated herein by reference.

### BACKGROUND OF THE INVENTION

Fluorescent real-time monitoring of polymerase chain reaction (“PCR”), a technique that employs intercalating agents or sequence-specific reporter probes to measure the concentration of amplified products as the reaction progresses, has been one of the most used methods of nucleic acid analysis since its introduction a decade ago. See Refs. 1, 2 and 3. The popularity of the technique stems not only from its sensitivity and quantitative resolution, but also the convenience of sample preparation, in which reagents are added to a regular PCR reaction mixture without additional pre or post-processing. See Ref. 4. However, the need for optical components for detection and optional quantification can limit the scalability and robustness of the measurement for miniaturization and field-uses, and the addition of external reagents can require extensive optimization or induce inhibitor effects. See Ref. 5.

Various non-optical label-free DNA sensing methods have been developed to quantify nucleic acids based upon their intrinsic properties. See Refs. 6-11. However, typically, these sensing techniques measure the hybridization of free, single-stranded targets in solution to immobilized single-stranded complementary probes, thus limiting their applicability in the detection and optional quantification of double-stranded nucleic acid. Since PCR generates double-stranded DNA (dsDNA), extra steps are necessary to generate single-stranded DNA (ssDNA) or RNA for hybridization. For example, sample preparation can require heating followed by rapid cooling of the product to prevent renaturation, or in-vitro transcription of double-stranded products to generate single-stranded RNA. See Ref. 12. These additional steps increase the complexity of tasks. that require repetitive assays such as real-time quantitative PCR. Repeated analysis and rinsing of the DNA capturing probe layer may damage the layer, thereby reducing its sensitivity. See Ref. 10. Furthermore, this feature hinders a sequential analysis of double-stranded nucleic acid at various stages of the amplification process or reaction.

Therefore, there exists a need in the art for improved methods for detection and optional quantification of double-stranded nucleic acids.

### SUMMARY

The present invention provides a method and apparatus for label-free detection of double-stranded nucleic acid. The method and apparatus maintains its sensitivity and is capable of a sequential analysis of amplified double-stranded nucleic acid, e.g., accumulated during multiple cycles of PCR in the presence of unprocessed PCR reagents. In various embodiments, the invention provides for a method and apparatus for label-free quantification of a double-stranded nucleic acid concentration.

In one aspect, the present invention is a method for the detection and optional quantification of a double-stranded nucleic acid comprising binding first layer comprising a charged species to a sensing surface, wherein the sensing surface was an associated first charge, and, wherein the first layer confers to the sensing surface a second charge or a neutral charge on a net basis, performing at least one cycle of DNA amplification to produce a double-stranded nucleic acid, and measuring interactions between the first layer and the double-stranded nucleic acid after the at least one cycle of DNA amplification.

In another aspect, the method for the detection and optional quantification of a double-stranded nucleic acid is cyclical and further comprises binding a second (third, fourth, fifth, . . . etc.) layer comprising a charged species to the double-stranded nucleic acid adjacent to and on top of the first (second, third, fourth, . . . etc. respectively) layer, and measuring interactions between the second (third, fourth, fifth, . . . etc. respectively) layer and the double-stranded nucleic acid introduced after a second (third, fourth, fifth, . . . etc. respectively) cycle of DNA amplification. For example, the present invention may further comprise binding a third layer comprising a charged species to the double-stranded nucleic acid adjacent to and on top of the second layer, and measuring interactions between the third layer and the double-stranded nucleic acid introduced after a third cycle of DNA amplification. In another aspect, the DNA amplification process is PCR.

In yet another aspect, the present invention is an apparatus for the detection and optional quantification of a double-stranded nucleic acid comprising a first chamber containing a sensing surface with an associated first charge; a first layer comprising a charged species, wherein the first layer material is bound to the sensing surface, and wherein the first layer has an associated second charge opposite to the first charge so that the first layer and sensing surface together create a second charge or neutral charge on a net basis; a second chamber for performing DNA amplification reactions; a means for removing a sample of double-stranded nucleic acid from the second chamber after at least one cycle of DNA amplification and introducing the sample into the first chamber. The apparatus may further comprise a measurement circuit operatively connected to the first chamber, for measuring a property of the interaction between the first layer and the double-stranded nucleic acid introduced into the first chamber.

In yet another aspect, the apparatus for the detection and optional quantification of a double-stranded nucleic acid operates in a cyclical manner, wherein the material for forming the second (third, fourth, fifth, . . . etc.) layer is taken from a reservoir comprising material for forming the layer, and is introduced to the first chamber in between cycles of DNA amplification. Each new layer is adjacent to the double-stranded nucleic acid introduced into the first chamber from a previous DNA amplification cycle. Interactions between the second (third, fourth, fifth, . . . etc. respectively) layer and the double-stranded nucleic acid after a second (third, fourth, fifth, . . . etc/) cycle of DNA amplification is measured. In other aspects, the apparatus further comprises a control means for controlling the introduction of the sample containing double-stranded nucleic acid and layer material to the first chamber and the DNA amplification cycles or reactions.

### BRIEF DESCRIPTION OF CERTAIN PREFERRED EMBODIMENTS OF THE INVENTION

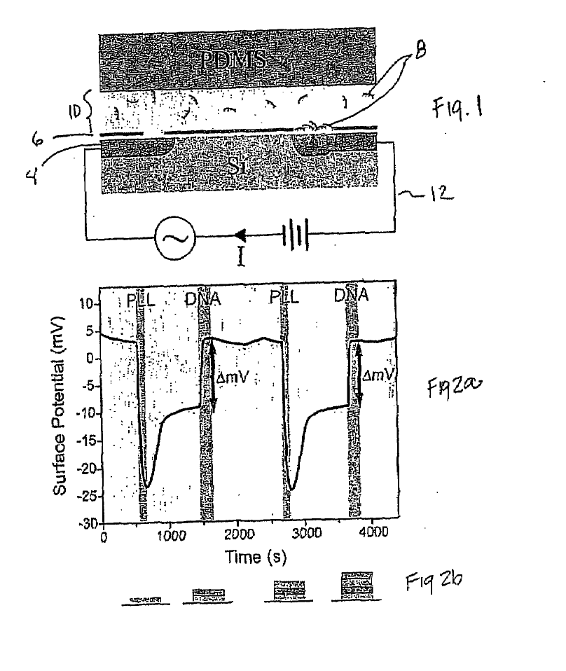

FIG. 1. is a schematic diagram depicting chemical interactions occurring at a sensing surface;

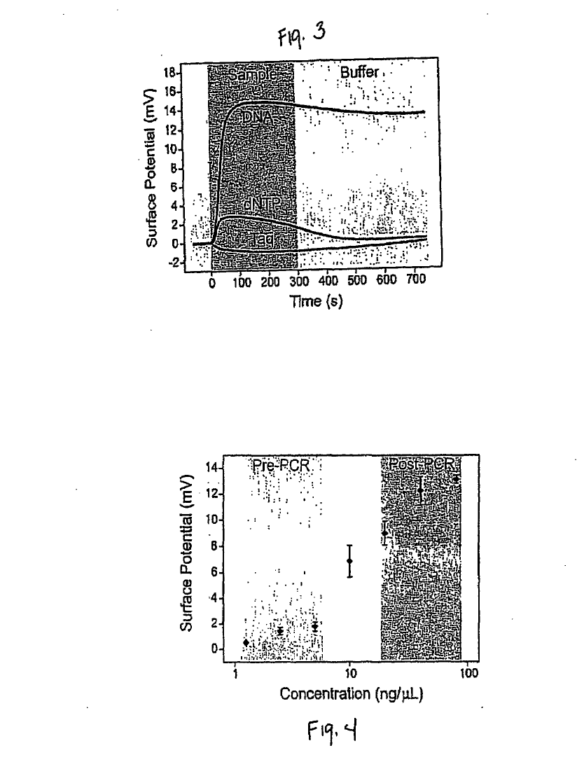

FIG. 2a. is a graph depicting the effect on the surface potential of the sensing surface upon alternately adding charged layers and double-stranded nucleic acid;

FIG. 2b. is a bar graph illustrating the expected increase in film thickness for alternating exposures of PLL and DNA;

FIG. 3. is a graph showing the sensitivity for individual components of a PCR mixture;

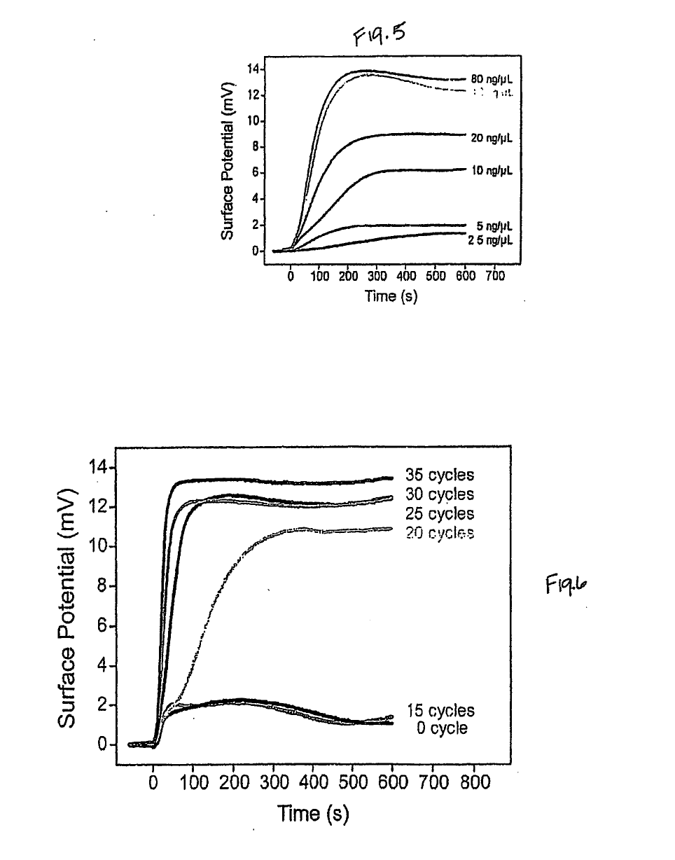

FIG. 4. is a graph showing DNA ladder at various dilutions used to obtain an average-case response for PCR products;

FIG. 5. is a graph illustrating the steady state response following sensor rinsing as a function of DNA concentration;

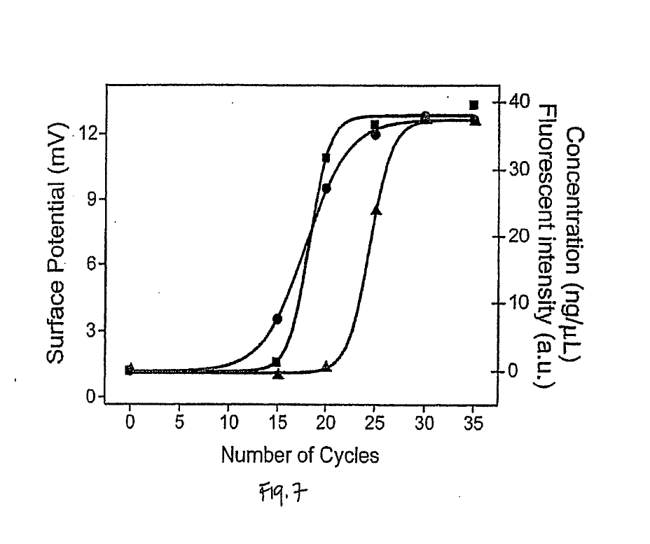

FIG. 6. is a graph showing the real-time electronic surface potential measurements for injections of PCR products; and

FIG. 7. compares the steady state response of electronic measurements and real-time monitoring of PCR.

### DETAILED DESCRIPTION OF CERTAIN PREFERRED EMBODIMENTS OF THE INVENTION

In one embodiment, the present invention is a method for the detection and optional quantification of a double-stranded nucleic acid comprising binding a first layer comprising a charged species to a sensing surface having an associated first charge, wherein the first layer confers to the sensing surface a second charge or a neutral charge on a net basis, performing at least one cycle of DNA amplification to produce a double-stranded nucleic acid, and measuring interactions between the first layer and the double-stranded nucleic acid after the at least one cycle of DNA amplification.

In another embodiment, the method for the detection and optional quantification of a double-stranded nucleic acid is cyclical and further comprises binding a second (third, fourth, fifth, . . . etc.) layer comprising a charged species to the double-stranded nucleic acid adjacent to and on top of the first (second, third, fourth, . . . etc. respectively) layer, and measuring interactions between the second (third, fourth, fifth, . . . etc. respectively) layer and the double-stranded nucleic acid introduced after a second (third, fourth, fifth, . . . etc. respectively) cycle of DNA amplification. For example, the present invention may further comprise binding a third layer comprising a charged species to the double-stranded nucleic acid adjacent to and on top of the second layer, and measuring interactions between the third layer and the double-stranded nucleic acid introduced after a third cycle of DNA amplification.

In one example, the DNA amplification process is PCR. PCR is a common method of DNA amplification, or creating copies of specific fragments of DNA. PCR rapidly amplifies a single DNA molecule into many billions of molecules. PCR is especially useful for searching out disease organisms that are difficult or impossible to culture, such as many kinds of bacteria, fungi, and viruses, because it can generate analyzable quantities of the organism's genetic material for identification. PCR looks directly for the virus's unique DNA, instead of the method employed by standard tests, which usually look for indirect evidence that the virus is present by searching for antibodies that the body has made against it. Refs. 23-30 and U.S. Pat. Nos. 5,333,675, 5,475,610, 5,656,493, and 6,814,934 disclose various methods and devices used in PCR, incorporated herein by reference. The method of the present invention may include the steps of disposing a charged layer onto the sensing surface, and measuring a property of that interaction of that layer with the double-stranded nucleic acid at a chosen time in the PCR amplification process. The charged (also known as the first) layer is electrostatically bound to the sensing surface. The charged/first layer and sensing surface together have an associated charge that will attract and bind the double-stranded nucleic acid. In another embodiment, the method for the detection and optional quantification of a double-stranded nucleic acid further comprises binding a second layer comprising a charged species to the double-stranded nucleic acid adjacent to and on top of the first layer, and measuring properties of that interaction between the second layer and the double-stranded nucleic acid after a another chosen time in the PCR amplification process.

The electronic sensing described and disclosed by the method and apparatus of the present invention offers a direct, label-free, double-stranded nucleic acid detection scheme based upon the optional quantification of total, double-stranded nucleic acid content in a reaction mixture. Layer-by-layer assembly is used to specifically detect amplified DNA. For example, in one embodiment, layer-by-layer assembly is used to specifically detect amplified DNA in PCR mixtures with a dynamic range relevant to the concentrations typically used for PCR. Furthermore, multilayer assembly of DNA and a charged polymer, for example, can tolerate underlying surface defects and contaminants, making such a system environment-tolerant and potentially feasible for field-uses. The present invention is capable of differentiating nucleic acid concentrations at various stages of PCR by producing a readout which resembles that of fluorescent measurements using intercalculating dyes in real-time PCR, but without their potential inhibitory artifacts. The sensing method and apparatus of the present invention enables a real-time PCR nucleic acid detection and optional quantification platform without the drawbacks associated with fluorescent optical systems. See Ref. 21.

When compared to other approaches of nucleic acid detection based on intercalating dyes such as Sybr Green I, the method and apparatus of the present invention offers similar sensitivity and selectivity, but the claimed method and apparatus are not prone to inhibition artifacts. The method and apparatus of the present invention detect and quantify double-stranded nucleic acid in its double-stranded state. In these embodiments, the present invention detects total amplified products indiscriminately and does not employ hybridization approaches. The electronic detection based on electrostatic association of polyelectrolytes offers several advantages over detection by hybridization. First, there is no need to denature the duplex double-stranded nucleic acid as in cases where immobilized single-stranded DNA is used as capturing probe. Second, the association rate resulting from electrostatic interactions between DNA and polyelectrolytes is up to three (3) orders of magnitude faster than that of hybridization. See Ref. 19. Third and as described above, the structural robustness of layer-by-layer deposition allows multilayers of up to hundreds of layers, and a fresh layer of a polyelectrolyte is deposited before every analytical step. This feature contrasts with techniques that rely on washing to regenerate probe surfaces for additional hybridization experiments. Such regenerations can require harsh conditions that cause damage to the functionalized surface, thereby reducing the sensitivity of the sensor by up to 20% between trials. See Ref. 10.

In other embodiments, the apparatus and method of the present invention detect and optionally quantify double-stranded nucleic acid amplified by ligation-based thermocycling approaches and other new PCR developments. In other examples, the apparatus and method of the present invention detect and optionally quantify double-stranded nucleic acid amplified by the alternative amplification processes to PCR, e.g., isothermal DNA amplification techniques such as real-time strand displacement amplification (“SDA”), rolling-circle amplification (“RCA”) and multiple-displacement amplification (“MDA”). Furthermore, the present invention contemplates the use of DNA amplification in the detection and optional quantification of non-DNA analytes.

In various embodiments, the sensing surface resides within a sample-containing region. The sample-containing region may be any structure suitable for fluid containment. The sensing surface has an associated first charge. This charge typically arises from the intrinsic properties of the material(s) comprising the sensing surface. For example, in one embodiment, the sensing surface comprises an oxide such as silicon dioxide. In such embodiment, the negatively charged oxygen molecules impart a net negative charge to the sensing surface. In other examples or embodiments, the material comprising the sensing surface is a thermally-grown oxide, chemically-grown oxide, or equivalents thereof, and may have a positive or neutral charge.

Underlying the sensing surface are one or more electrically responsive layers, which sense molecular interactions occurring at the sensing surface. In one embodiment, the measurement circuit detects and optionally quantifies the electrical response between the charged layers on the sensing surface and the double-stranded nucleic acid, and optionally the degree and/or character of the interaction. In another embodiment, the measurement circuit detects and optionally measures mass and/or thickness of the layer formation and DNA binding.

FIG. 1 is an exemplary illustration of a molecular level view of the interactions of various components at the sensing surface of the present invention. In one embodiment, the sensing surface 4 comprises charged oxygen molecules (not shown). Constituents of the first layer 6 comprising a charged species are introduced into a sample-containing region 10 and bind to the sensing surface 4. In another embodiment, the first layer 6 is positively charged and comprises, for example, a positively charged polymer. In yet another embodiment, the first layer 6 may comprise poly-L-lysine (“PLL”). DNA amplification will produce complimentary double-stranded nucleic acids 8. In one embodiment, the double-stranded nucleic acid 8 is negatively charged. Consequently, the double-stranded nucleic acid 8 binds electrostatically to the first layer 6 when introduced into the sample-containing region 10. The first layer may alternatively or further comprise other positively charged species such as histones, protamines, polyarginine, polyornithine, DEAE dextran, polybrene, and polyethylenimine.

The initial binding of the first layer 6, and the subsequent binding of the double-stranded nucleic acid 8 to the first layer 6, causes changes at the sensing surface 4 which can be measured and distinguished from one another by the measurement circuit 12. Electrical changes at the sensing surface 4 can be measured through capacitive, current, or voltage-based means. Mass and/or thickness changes may also be measured (e.g., through index of refraction). The magnitude of the change is correlated with the degree of interaction between the sensing surface 4 and the double-stranded nucleic acid 8.

It should be noted that multiple layers of polyelectrolyte are known to increase monotonically in thickness with alternating depositions of oppositely charged species and thus yield rising signals when measured by mass or optical index sensitive measurements. Various embodiments of the present invention reveal a markedly different result. The depositions of oppositely charged species (e.g., positively charged PLL and negatively charged double-stranded DNA) shows a cyclical pattern where the deposition of a given one charged species consistently results in a decrease of signal and the subsequent binding of an oppositely charged species results in an increase. The same trend is observed for many iterations of oppositely charged species, without noticeable degradation in the amplitude of the equilibrated signal.

Referring to one example of the present invention demonstrated in the graph of FIG. 2a, the initial introduction of positively charged PLL results in the reduction of the surface potential as the PLL bonds to a silicon dioxide sensing surface. When a negatively charged oligonucleotide is introduced, the surface potential rises as the oligonucleotide binds to the PLL and to the sensing surface. The second introduction of PLL demonstrates the same surface-potential-reducing effect as the first PLL introduction. The second introduction of the oligonucleotide once again increases the surface potential. Thus, this method of charge regulation through a charge-regulating layer and double-stranded nucleic acid is repeatable on the same device. FIG. 2b is a bar graph (not drawn to scale) illustrating conceptually the expected monotonic increase in film thickness for alternating exposures to PLL and double-stranded nucleic acid. However, as shown in FIG. 2a, cyclical patterns (increase for DNA and decrease for PLL) without a degradation in signal are observed for the electronic measurements of various embodiments of the present invention.

In yet another embodiment, the present invention is an apparatus for the detection and optional quantification of a double-stranded nucleic acid comprising a first chamber comprising a sensing surface with an associated first charge, a first layer comprising a charged species, wherein the first layer material is bound to the sensing surface, and wherein the first layer has an associated second charge opposite to the first charge so that the first layer and sensing surface together create a second charge or neutral charge on a net basis; a second chamber for performing DNA amplification reactions; a means for removing a sample of double-stranded nucleic acid from the second chamber after at least one cycle of DNA amplification and introducing the sample into the first chamber. The apparatus further comprises a measurement circuit operatively connected to the first chamber, for measuring at least one property of the interaction between the first layer and the double-stranded nucleic acid introduced into the first chamber. The apparatus may further comprise microfluidic channels, valves and pumps to enable or facilitate fluid transfer into, out of, and between the chambers of various embodiments of the present invention.

In another embodiment, the double-stranded nucleic acid is a product of an amplification process or procedure, such as ligation-based thermocycling approaches and other new PCR developments. In other examples, the apparatus of the present invention detects and optionally quantifies double-stranded nucleic acid amplified by isothermal DNA amplification techniques such as real-time strand displacement amplification (“SDA”), rolling-circle amplification (“RCA”) and multiple-displacement amplification (“MDA”). Furthermore, the present invention contemplates the use of DNA amplification in the detection and optional quantification of non-DNA analytes.

The interaction between the layers and the amplification product is detected and optionally quantified in real-time. In yet another embodiment, the apparatus for the detection of a double-stranded nucleic acid operates in a cyclical manner, wherein the material for forming the second (third, fourth, fifth, . . . etc.) layer is taken from a reservoir comprising material for forming the layer, and is introduced to the first chamber in between cycles of DNA amplification. Each new layer is adjacent to the double-stranded nucleic acid introduced into the first chamber from a previous DNA amplification cycle. Interactions between the second (third, fourth, fifth, . . . etc., respectively) layer and the double-stranded nucleic acid after a second (third, fourth, fifth, . . . etc.) cycle of DNA amplification is measured.

In still other embodiments, the apparatus further comprises a plurality of reservoirs or equivalents thereof (e.g., discrete zones, channels, contained portions or regions, cavities, or any other areas that can be isolated from the sensing surface while the layers are being deposited). These additional reservoirs may comprise any substance or material intended to be added to the sample-containing region, or the first and/or second chambers, such as wash reagents or pre-selected primers. In addition, the apparatus may comprise various means for introducing the substance or material from the reservoirs into the sample-containing region, or the first and/or second chamber (or any other reservoir or chamber located within or works in conjunction with the apparatus of the present invention). The apparatus may comprise pumps and valves for moving material around the apparatus. In one embodiment, wherein the apparatus is located on a microchip, the apparatus comprises pumps and valves that work similar to that described in Refs. 30 and 31, herein incorporated by reference, to enable the movement of various materials in and around the apparatus of the present invention. For example, wherein the apparatus comprises an X layer comprising a charge species, the X layer material may have been provided to the sample-containing region to bind to the sensing surface by a pump and/or valve system which removed the X layer material from a reservoir or chamber external to the sample-containing region.

The apparatus may further comprise a control means for controlling the introduction of any material (e.g., the sample containing double-stranded nucleic acid and layer material) to the chambers and/or the sample-containing region, as well as the DNA amplification cycles or reactions. The control means may comprise a computer-readable medium with software to process at least one step of the claimed methods of the present invention. The control means may be used to control the operation of the apparatus, e.g, introducing the double-stranded nucleic acid into the first chamber and performing various cycles of amplification. The control means may also include a means for programming the control means or apparatus, and optionally further comprise a display means for providing a readout of information from the apparatus. For example, the display means may comprise a light-emitting diode that lights up when a certain level of double-stranded nucleic acid has been detected. The display may further display the cycle or reaction number of the amplification process which produced a threshold amount or level of double-stranded nucleic acid.

For example, after one cycle of PCR is performed and the double-stranded nucleic acid is measured, the control means may instruct the apparatus to introduce another charged species later into the first chamber, wherein the newly-added layer has an associated second charge opposite to the charge of the double-stranded nucleic acid. The newly-added layer together with the previously introduced double-stranded nucleic acid have a second charge or a neutral charge on a net basis. Another cycle or several cycles of PCR are performed and the double-stranded nucleic acid is again introduced into the first chamber and measured. The measurement circuit measures interactions between the newly-added layer and the double-stranded nucleic acid after the second or several additional PCR cycles. The display lights up when the apparatus measures a predetermined amount of double-stranded nucleic acid, or a specific type of interaction.

Such a system as described and disclosed above is robust and can be manufactured using standard microelectronics fabrication technologies for on-chip applications. For example, since PCR protocols can be shortened on a microfabricated silicon device due to its low thermal mass and high thermal conductivity, in one aspect, the present invention can genotype single cells on an integrated, microfluidic, portable device in a short amount of time (e.g., minutes). See Ref. 22. The method and apparatus of the present invention enables electronic monitoring of PCR amplification on a chip (a prerequisite for the miniaturization and parallelization of diverse PCR-based technologies ranging from DNA computation to in vitro nucleic acid evolution).

In various other embodiments of the present invention, the apparatus is a kit, wherein the kit may comprise one or more of the mentioned components listed in this specification or used by the apparatus of the claimed invention, including equivalents thereof.

It should be noted that where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

### EQUIVALENTS

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference.

The following examples contain important additional information, exemplification and guidance that can be adapted to the practice of this invention in its various embodiments and the equivalents thereof.

### EXEMPLIFICATION

The method of this invention can be understood further by the examples that illustrate some of the ways by which the inventive apparatus may be constructed, and the method may be practiced. It will be appreciated, however, that these examples do not limit the invention. Variations of the invention, now known or further developed, are considered to fall within the scope of the present invention as described herein and as hereinafter claimed.

### Example 1

### Fabrication of Device for Label-Free Detection of Double-Stranded Nucleic Acids

As shown in FIG. 1, one embodiment of the present invention demonstrates quantitative label-free monitoring of product formation in an unprocessed PCR mixture using a microelectronic sensor based on silicon field-effect. High sensitivity to the intrinsically charged PCR product 8 was achieved by depositing a thin layer of PLL 6 on the sensing surface 4. In this configuration, the sensor could quantitatively and reproducibly differentiate concentrations of DNA in the PCR relevant range of approximately 1-80 ng/μL. After measuring a particular concentration, the sensor was readily recovered without degradation of its sensitivity by depositing another layer of PLL 6 on the sensor surface 4. Therefore, the technique was capable of sequentially analyzing PCR products 8 at various stages of the reaction through layer-by-layer assembly. Furthermore, this method embodiment was performed in microfluidic channels with nanoliter volumes.

The silicon field-effect sensor was at least one oxide-based electrolyte-insulator-semiconductor (“EIS”) capacitor(s) fabricated on planar silicon substrates. The sensor consisted of a charge sensitive region encapsulated by polydimethylsiloxane (“PDMS”) microfluidics molded by soft lithography. See Ref. 13. Processing began with 6″ n-type (phosphorus doped) 20-50 Ω-cm silicon substrates. Conventional photolithography was used to define implants of active sensor areas (lightly-doped p-type), conductive traces (heavily-doped p-type), and an isolating ground plane (heavily-doped n-type). After annealing, a relatively uniform doping level of 10¹⁵ atoms/cm² down to 0.8 μm silicon-rich nitride was then deposited. Metal contact holes and 80×80 μm2 sensor areas were then etched in the silicon nitride in a single step. Finally, 30 nm Ti and 1 μm Au were evaporated on the substrate as conductive traces and patterned using a liftoff process. PDMS microfluidic channels were molded from an SU-8 master, and inlets were punched with a 19-gauge needle.

The silicon device was submerged in hot piranha solution (1:3 30% H₂0₂ in H₂SO₄) for 5 minutes. The silicon chip was then rinsed with deionized water, dried with nitrogen, aligned, and hermetically bonded to PDMS. Buffered oxide etchant (7:1 H₂O:HF) was introduced into the microfluidic channel for 20 seconds. PCR buffer was flowed through the channel overnight to equilibrate the sensor. The surface preparation procedure left a thin native oxide on the sensor surface 4.

### Example 2

### Monitoring Formation of Polyelectrolyte Multilayers

Functionalization of the surface was performed by exposing the sensor surface to 0.2 mg/ml poly-L-lysine hydrobromide (PLL, MW 15,000-30,000, Sigma) in PCR buffer for 3 minutes then rinsing off the unbound species with PCR buffer for 10-15 minutes. A DNA response curve was generated by diluting stock 50 bp DNA ladder (New England BioLabs, Ipswich, Mass.) to various concentrations with PCR buffer. Human genomic DNA was purchased from Maxim Biotech, Inc. (South San Francisco, Calif.). For all binding experiments on PLL-coated surfaces, the sensors were exposed to analytes for 5 minutes followed by a 5 minute wash during which time the signal is recorded.

The sensor behaved as a variable capacitor whose impedance value was sensitive to the charge density of surface bound molecules. See Refs. 14 and 15. The sensor was used to monitor the formation of polyelectrolyte multilayers through alternating injections of PLL, a positively charged polypeptide, and DNA, which carries two negative charges per base pair. The thickness of polyelectrolyte multilayers is known to increase with alternating depositions of oppositely charged species due to electrostatic associations thus yielding rising signals when measuring mass or thickness. See Refs. 16-19. However, as shown in FIG. 2a and 2b, polyelectrolyte multilayer deposition on the intrinsically negative charged field-effect sensor surface revealed a markedly different behavior. The deposition of a positively charged polymer 6 consistently resulted in a decrease of signal and the subsequent adsorption of a negatively charged species 8 resulted in an increase. The trend was observed for depositions of many layers without noticeable degradation in the amplitude of the equilibrated signal, indicating that the overcompensated surface charge at the top layer was effectively propagated to the sensor surface. This feature allowed the performance of multiple rounds of DNA measurements by resetting the baseline signal by re-depositing PLL onto the surface. At the same time, the additive surface regeneration process ensured that the surface was saturated by positive charges and the binding capacity did not degrade for multiple DNA analyses.

### Example 3

### Response of Sensor to Different DNA Concentrations

The sensor was tested for its response to DNA at concentrations in the range relevant to PCR conditions. DNA ladder of lengths between 50 bp to 1350 bp (representative of various PCR product sizes) were chosen to obtain the DNA mass concentration response of the sensor. The DNA mass concentration was empirically determined using Labchip kits wherein product concentrations between 20 and 50 ng/μL are obtained from various saturated PCR experiments. The dependence of surface potential change on DNA concentrations between 2.5 and 80 ng/μL was tested. The device was most sensitive to DNA concentrations between 10 ng/μL and 40 ng/μL, a range relevant to PCR quantifications.

The measurement method has been previously reported in detail. See Ref. 6. Briefly, a 4 kHz, 50 mVpp ac voltage was delivered to the on-chip gold signal electrode. The resulting alternating current through the sensor was amplified and converted by a lock-in amplifier to a dc voltage that was proportional to the capacitance of the depletion region. The capacitance of the depletion region was a function of the potential difference between the electrolyte-insulator interface and p-type sensor region in the EIS structure. Before each experiment, the bias potential applied to the sensor was set at a level where the slope and linearity of the output versus sensor bias voltage curve are maximized. See Ref. 15. The relative surface potential response of the sensor as a function of the lock-in amplifier output was calibrated by applying a bias step to the sensor. All surface potential values were relative. Constant fluid flow of 10 μL/min was used for all measurements. Analytes were injected by an autosampler (Hitachi High Technologies America), and signals were recorded at 10 Hz.

The electronic detection of DNA/PLL multilayers is useful for PCR product analysis if the measurement is primarily sensitive to the products of interest in a PCR mixture. FIG. 3 shows the electronic measurement of individual components of a PCR reaction mixture according to this embodiment of the method and apparatus of the present invention. In order for the electronic measurement of multilayer formation to be feasible for double-stranded nucleic acid analysis, it must primarily produce a signal from the double-stranded nucleic acid in a PCR reaction mixture. As shown in FIG. 3, individual components of a PCR reaction mixture (e.g., Taq and dNTP) that were injected onto a PLL-coated surface of one embodiment of the invention, result in insignificant alterations of surface potential when compared with the surface potential change due to a double-stranded nucleic acid injection. To characterize this, the PLL-coated surface of the sensor was exposed to individual components present in the PCR mixture: Taq polymerase, nucleotide monomers (deoxynucleotide triphosphate or “dNTP”), and DNA including primers, templates, and PCR products. The components were introduced at the same concentrations used for PCR in order to quantify their corresponding surface potential response. Injections of Taq polymerase alone did not result in a permanent change in signal after subsequent rinsing. In contrast, 40 ng/μL of dsDNA ladder resulted in a clearly resolvable baseline shift.

The ability of the sensor to resolve different concentrations of DNA in the range relevant to PCR conditions was also tested. A range of DNA ladder lengths (50 bp to 1350 bp) were selected, representative of various amplicon sizes. The sensor's response to each component was characterized at the concentrations used for the PCR experiments. In particular, and as shown in FIGS. 4 and 5, the sensor's response to 0.4 μM forward and reverse primers, 2 ng/μL genomic DNA (equivalent to 100 ng DNA template in 50 μL of PCR buffer), and 30 ng/μL purified amplification product yielded average surface potential changes of 1 mV, 1 mV, and 10 mV, respectively. This result indicated that even though some of the primers and templates in a PCR mixture will produce background signals, the product is expected to contribute most significantly to the overall sensor readout by the later cycles of a typical PCR reaction. Furthermore, based upon this data, several inferences can also be made about the sensitivity of the electronic sensor to DNA length. The 0.4 μM 20 bp forward and 22 bp reverse primers account for a mass concentration of approximately 5 ng/μL and resulted in an output similar to that of a DNA ladder at the same concentration. In addition, 2 ng/PL of genomic DNA, 90% of which was expected to be larger than 50 kbp according to the product manual, produced a signal comparable to that of 2 ng/μL DNA ladder. Finally, 3 ng/μL of purified product yielded a surface potential change of 10 mV which is in between the average surface potential measurements for 20 ng/μL and 40 ng/μL ladders. These comparisons showed that for concentrations relevant to PCR, the measurement method is sensitive primarily to the mass concentration of DNA rather than to its length.

### Example 4

### Monitoring Product Generation During a PCR Reaction

The types of nucleic acids in a PCR mixture include primers, templates, and products. For this example, forward primer, 5′ ATC AAG CAG CCA TGC AAA TG 3′, and reverse primer, 5′ CCT TTG GTC CTT GTC TTA TGT C 3′, were used to amplify a 291 base pair (bp) fragment of the HIV-1 GAG gene. The PCR buffer consisted of 10 mM Tris-HCl (pH 8.3), 20 mM KCl, 2 mM MgCl₂. The reaction mixture included the PCR buffer, 0.1 mM each of dNTPs, 0.4 μM each of forward and reverse primers, 5 U Taq polymerase (New England BioLabs, Ipswich, Mass.), and positive control templates (1 ng/mL) (Maxim Biotech, South San Francisco). For real-time PCR, Sybr Green I (Molecular Probes, Eugene, Oreg.), was added to the PCR mixture using a 10,000-fold dilution of the stock solution. The PCR was performed for 35 cycles of 90° C. for 25 seconds, 53° C. for 30 seconds, and 72° C. for 50 seconds, or stopping at earlier cycles when necessary. For control experiments, QIAquick PCR Purification Kit (Qiagen, Valencia, Calif.) was used to isolate the PCR products from saturated PCR reactions.

A segment of the HIV-1 GAG gene was amplified and the products were analyzed at various stages of the reaction. FIG. 6 illustrates the time-course response of the electrical detector. Reaction mixtures terminating after different number of cycles (0, 15, 20, 25, 30, 35) were introduced to the electronic sensors for five (5) minutes, followed by a five (5) minute wash. For control experiments, 40 ng/μL DNA ladder was injected to the electronic sensors twice, before and after the analysis of PCR products, and resulted in 13.3 mV and 13.0 mV change in surface potential, respectively. Amplifying from a starting concentration of 1 ng/μL, real-time Sybr Green I fluorescence readout showed a marked increase after approximately 20 cycles of amplification. Electronic measurements of PCR experiments terminating after various cycles also showed an increase in output after the 15th cycle, but the steepest rise was registered between the 15th and 20th cycle, compared to the 20th and 25th cycle for the Sybr Green measurement. The PCR products used for the electronic measurements were also separated and quantified using Labchip kits. FIG. 7 shows the comparison of end-point measurements by optical detection with an intercalating dye, gel electrophoresis, and electronic detection. As shown in the figure, the product concentrations as measured by the Labchip kits correspond with the electronic results. Both sets of data show the largest increase between the 15th and 20th cycle, indicating the electronic readouts were representative of product concentrations. The discrepancy of the Sybr Green I measurement might be explained by the fluorescent reagent partially inhibiting the PCR reaction. A total inhibition of PCR reaction was observed when three times (3×) the recommended concentration (10,000× dilution of stock solution) of Sybr Green I dye was included in the PCR mixture at low starting template concentrations which otherwise yielded a positive amplification. As a control, 40 ng/μL of DNA ladder was introduced to the electronic sensor before and after analyzing the series of PCR products. Similar results were observed, confirming that the sensitivity of the sensor was preserved throughout the measurements. Furthermore, since electronic PCR generates a curve analogous to that of a fluorescent real-time PCR system, the same method may be used to achieve a similar range for nucleic acid quantification. For example, the initial copy number may be correlated with the number of PCR cycles required for the curve to exceed a threshold value.

### REFERENCES

1. Higuchi, R., Fockler, C., Dollinger, G. & Watson, R. Kinetic PCR analysis: real-time monitoring of DNA amplification reactions. Biotechnology 11, 1026-1030 (1993).
2. Tyagi, S. & Kramer, F. R. Molecular beacons: probes that fluoresce upon hybridization Nat. Biotechnol. 14, 303-308 (1996).
3. Heid, C. A., Stevens, J., Livak, K. J. & Williams, P. M. Real time quantitative PCR. Genome Res. 6, 986-994 (1996).
4. Klein, D. Quantification using real-time PCR technology: applications and limitations. TRENDS Mol. Med. 8, 257-260 (2002).
5. Nath, K., Sarosy, J. W., Hahn, J. & Di Como, C. J. Effects of ethidium bromide and SYBR Green I on different polymerase chain reaction systems. J. Biochem. Biophys. Methods 42, 15-29 (2000).
6. Fritz, J., Cooper, E. B., Gaudet, S., Sorger, P. K. & Manalis, S. R. Electronic detection of DNA by its intrinsic molecular charge. Proc. Natl. Acad. Sci. USA 99, 14142-14146 (2002).
7. Fritz, J., Baller, M. K., Lang, H. P., Rothuizen, H., Vettiger, P., Meyer, E., Güntherodt, H.-J., Gerber, C. & Gimzewski, J. K. Translating Biomolecular Recognition into Nanomechanics. Science 288, 316-318 (2000).
8. Woods, S. J. DNA-DNA hybridization in real time using BIAcore. Microchem. J. 47, 330-337 (1993).
9. Nelson, B. P., Grimsurd, T. E., Liles, M. R., Goodman, R. M. & Corn, R. M. Surface Plasmon Resonance Imaging Measurements of DNA and RNA Hybridization Adsorption onto DNA Microarrays. Anal. Chem. 73, 1-7 (2001).
10. Fan, C., Plaxco, K. W. & Heeger, A. J. Electrochemical interrogation of conformational changes as a reagentless method for the sequence-specific detection of DNA. Proc. Natl. Acad. Sci. USA 100, 9134-9137 (2003).
11. Armistead, P. M. & Thorp, H. H. Modification of indium tin oxide electrodes with nucleic acids: detection of attomole quantities of immobilized DNA by electrocatalysis. Anal. Chem. 76, 3764-3770 (2000).
12. Innis, M. A., Gelfand, D. H., Sninsky, J. J., White, T. J., PCR protocols: a guide to methods and applications (Academic Press, San Diego; 1990).
13. Whitesides, G. & Xia, Y. Soft lithography. Angew. Chem. Int. Edn Engl. 37, 550-575 (1998).
14. Bousse, L. & Bergveld, P. On the impedance of silicon dioxide/electrolyte interface. J. Electroanal. Chem. 152, 25-39 (1983).
15. Cooper, E. B., Fritz, J., Wiegand, G., Wagner, P. & Manalis, S. R. Robust microfabricated field-effect sensor for monitoring molecular adsorption in liquids. Appl. Phys. Lett. 79, 3875-3877 (2001).
16. Decher, G. Fuzzy Nanoassemblies: Toward Layered Polymeric Multicomposites. Science 277, 1232-1237 (1997).
17. Lvov, Y., Decher, G. & Sukhorukov, G. Assembly of thin films by means of successive deposition of alternate layers of DNA and poly (allylamine). Macromolecules 26, 5396-5399 (1994).
18. Pei, R., Cui, X., Yang, X., Wang, E. Assembly of Alternating Polycation and DNA Multilayer Films by Electrostatic Layer-by-Layer Adsorption. Biomacromolecules 2, 463-468 (2001).
19. Wink, T., de Beer, J., Hennink, W. E., Bult, A. & van Bennekom, W. P. Interaction between plasmid DNA and cationic polymers studied by surface plasmon resonance spectrometry. Anal. Chem. 71, 801-805 (1999).
20. Picart, C., Mutterer, J., Richert, L., Luo, Y., Prestwich, G. D., Schaaf, P., Voegel, J.-C. & Lavalle, P. Molecular basis for the explanation of the exponential growth of polyelectrolyte multilayers. Proc. Natl. Acad. Sci. USA 99, 12531-12535 (2002).
21. Burns, M. A., Johnson, B. N., Brahmasandra S. N., Handique, K., Webster, J. R., Krishnan, M., Sammarco, T. S., Man, P. M., Jones, D., Heldsinger, D., Mastrangelo, C. H. & Burke, D. T. An Integrated Microfabricated DNA Analysis Device. Science 282, 484-487 (1998).
22. Northrup, M. A., Ching, M. T., White, R. M. & Watson, R. T. DNA amplification with a microfabricated reaction chamber. Transducers '93, 924-927 (1993).
23. Saiki R, K., Scharf S, Faloona F, Mullis K. B, Horn G. T, Erlich H. A, Arnheim N. Enzymatic amplification of beta-globin genomic sequences and restriction site analysis for diagnosis of sickle cell anemia. Science, 230 (4732): 1350-4 (Dec. 20, 1985).
24. Mullis K. B, Faloona F. A, Scharf S, Saiki R. K, Horn G, Erlich H. A. Specific enzymatic amplification of DNA in vitro: the polymerase chain reaction. Cold Spring Harbor Symposia on Quantitative Biology (1986).
25. Scharf S. J, Horn G. T, Erlich H. A. Direct cloning and sequence analysis of enzymatically amplified genomic sequences. Science, 233(4768): 1076-8 (Sep. 5, 1986).
26. Saiki R. K, Bugawan T. L, Horn G. T, Mullis K. B, Erlich H. A. Analysis of enzymatically amplified beta-globin and HLA-DQ alpha NDA wth allele-specific oligonucleotide probes. Nature, 324(6093): 163-3 (Nov. 13-19, 1986).
27. Mullis K. B, Faloona F. A. Specific synthesis of DNA in vitro via a polymerase-catalyzed chain reaction. Methods in Enzymology, 155:335-50 (1987).
28. Saiki R. K, Gelfand D. h, Stoffel S, Scharf S. J, Higuchi R, Horn G. T, Mullis K. B, Erlich H A. Primer-directed enzymatic amplification of DNA with a thermostable DNA polymerase. Science, 239(4839): 487-91 (Jan. 29, 1988).
29. Lawyer F. C, Stoffel S, Saiki R. K, Myambo K, Drummond R, Gelfand D. H. Isolation, characterization, and expression in Escherichia coli of the DNA polymerase gene from Thermus aquaticus. Journal of Biological Chemistry, 264(11): 6427-37 (Apr. 15, 1989).
30. Guyer R. L, Koshland D. E, Jr. The molecule of the year. Science, 246(4937): 1543-6 (Dec. 22, 1989).
31. Unger M A, Chou H P, Thorsen T, Scherer A, Quake S R. Monolithic microfabricated valves and pumps by multilayer soft lithography. Science 2000; 288: 5463: 113-6.
32. Grover, W. H., Skelley, A. M., Liu, C. N., Lagally, E. T., and Mathies, R. A. Monolithic membrane valves and diaphragm pumps for practical large-scale integration into microfluidic devices, sensors & actuators. B, 89, 315-323 (2003).

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. Furthermore, where the claims recite an apparatus, it is to be understood that methods of using the apparatus as described in any of the claims reciting methods are also disclosed, unless it would be evident to one of ordinary skill in the art that a contradiction or inconsistency would arise. It is also to be understood that where the claims recite an apparatus that has particular features or characteristics, the invention encompasses an apparatus comprising means for implementing such features or characteristics. In addition, it is to be understood that any particular embodiment of the present invention that falls within the prior art may be explicitly excluded from the claims. Since such embodiments are deemed to be known to one of ordinary skill in the art, they may be excluded even if the exclusion is not set forth explicitly herein.

What is claimed is:

1. A method for detecting and optionally quantifying a double-stranded nucleic acid comprising:
(a) binding a first layer comprising a charged species to a sensing surface, wherein the sensing surface has an associated first charge, and wherein the first layer confers to the sensing surface a second charge or a neutral charge on a net basis;
(b) performing at least one cycle of DNA amplification to produce a double-stranded nucleic acid; and
(c) measuring a property of an interaction between the first layer and the double-stranded nucleic acid.

2. The method of claim 1, wherein the second charge substantially neutralizes the first charge.

3. The method of claim 1, wherein the second charge reverses the first charge.

4. The method of claim 1, wherein the first charge is a negative charge and wherein the second charge is positive.

5. The method of claim 1, wherein the first charge is a positive charge and wherein the second charge is negative.

6. The method of claim 1, wherein the first layer comprises a charged polymer, organic polycation, non-polymeric cation, or an inorganic material.

7. The method of claim 1, wherein step (b) comprises performing at least one cycle of polymerase chain reaction.

8. The method of claim 1, wherein step (b) comprises performing at least one cycle of isothermal DNA amplification, real-time strand displacement, rolling-circle amplification, or multiple-displacement amplification.

9. The method of claim 1, wherein the first layer comprises polylysine.

10. The method of claim 1, wherein the first layer comprises one or more charged species selected from the group consisting of histones, protamines, polyarginine, polyomithine, DEAE dextran, polybrene, and polyethylenimine.

11. The method of claim 1, wherein the property of the step (c) is measured in the presence of at least one other component of the at least one amplification cycle.

12. The method of claim 11, wherein at least one component of the amplification reaction is an enzyme, dNTP, primer, or template.

13. The method of claim 1, comprising providing a sensing surface having an associated first charge.

14. The method of claim 1, comprising electrostatically binding the first layer to the sensing surface, wherein the binding produces an electrical response.

15. The method of claim 14, comprising correlating a magnitude of the electrical response with a degree of interaction between the first layer and the double-stranded nucleic acid.

16. The method of claim 1, wherein the interaction between the first layer and the double-stranded nucleic acid alters an electronic property at the sensing surface, the alteration being indicative of the interaction.

17. The method of claim 1, wherein the interaction between the first layer and the double-stranded nucleic acid alters a capacitance property, the alteration being indicative of the interaction.

18. The method of claim 17, comprising immersing the sensing surface and first layer in an electrolyte solution, and measuring at least a portion of the capacitance property change between the first layer and the double-stranded nucleic acid.

19. The method of claim 16, wherein the electronic property is at least one of capacitance, conductance, impedance, resistance, current, voltage, or electric field intensity.

20. The method of claim 1, comprising:
(d) binding a second layer comprising a charged species to the double-stranded nucleic acid adjacent to the first layer;
(e) performing at least one cycle of DNA amplification to produce a double-stranded nucleic acid; and
(f) measuring a property of an interaction between the second layer and the double-stranded nucleic acid produced by step (e).

21. The method of claim 1, comprising:
(g) binding an X layer comprising a charged species to the double-stranded nucleic acid adjacent to the X−1 layer;
(h) performing a Y cycle of DNA amplification to produce a double-stranded nucleic acid; and
(i) measuring a property of an interaction between the X layer and the double-stranded nucleic acid introduced after a Y cycle of DNA amplification, wherein X and Y are integers, and wherein X is greater than or equal to two and Y is greater than one.

22. The method of claim 21, further comprising repeating steps (g) through (i), and incrementing X and Y respectively by at least one integer each time steps (g) through (i) are repeated.

23. The method of claim 21, wherein the X layer is a charged polymer, organic polycation, non-polymeric cation, or an inorganic material.

24. The method of claim 21, wherein the X layer comprises polylysine.

25. The method of claim 21, wherein the X layer comprises one or more charged species selected from the group consisting of histones, protamines, polyarginine, polyornithine, DEAE dextran, polybrene, and polyethylenimine.

26. The method of claim 21 wherein the property of step (c) is measured in the presence of at least one other component of the amplification reaction.

27. The method of claim 21, wherein at least one other component of the amplication reaction is an enzyme, dNTP, primer or template.

28. The method of claim 21, comprising providing a sensing surface having an associated first charge.

29. The method of claim 21, comprising electrostatically binding the X layer to the double-stranded nucleic acid bound to the X−1 layer, wherein the binding produces an electrical response.

30. The method of claim 29, comprising correlating a magnitude of the electrical response with a degree of interaction between the X layer and the double-stranded nucleic acid introduced after a Y cycle of DNA amplification.

31. The method of claim 21, wherein the interaction between the X layer and the double-stranded nucleic acid alters an electronic property at the sensing surface, the alteration being indicative of the interaction.

32. The method of claim 21, wherein the interaction between the X layer and the double-stranded nucleic acid alters a capacitance property, the alteration being indicative of the interaction.

33. The method of claim 32, comprising immersing the sensing surface and X layer in an electrolyte solution, and measuring at least a portion of a capacitance property change between the first layer and the double-stranded nucleic acid.

34. The method of claim 31, wherein the electronic property is at least one of capacitance, conductance, impedance, resistance, current, voltage, and electric field intensity.

35. The method of claim 21, wherein the sensing surface is located within a first chamber and the at least one cycle of DNA amplification is performed in a second chamber.

36. The method of claim 35, wherein the method comprises introducing a sample from the second chamber into the first chamber.

37. The method of claim 21, comprising cleaning the sensing surface with a cleaning agent.

38. An apparatus for detecting and optionally quantifying a double-stranded nucleic acid comprising:
a first chamber comprising a sensing surface with an associated first charge;
a first layer comprising a charged species, wherein the first layer is bound to the sensing surface, and wherein the first layer has an associated second charge opposite to the first charge so that the first layer and sensing surface together create a second charge or neutral charge on a net basis;
a second chamber for performing DNA amplification reactions;
a means for removing a sample comprising double-stranded nucleic acid from the second chamber after at least one cycle of DNA amplification and introducing the sample into the first chamber; and
a measurement circuit operatively connected to the first chamber, for measuring a property of an interaction between the first layer and the double-stranded nucleic acid introduced into the first chamber after the at least one cycle of DNA amplification.

39. The apparatus of claim 38, wherein the second charge substantially neutralizes the first charge.

40. The apparatus of claim 38, wherein the first charge is a negative charge and wherein the second charge is positive.

41. The apparatus of claim 38, wherein the first charge is a positive charge and wherein the second charge is negative.

42. The apparatus of claim 38, further comprising:
a reservoir, wherein the reservoir comprises material for forming a first layer; and
a means for providing the material for forming the first layer into the first chamber.

43. The apparatus of claim 38, further comprising:
a plurality of reservoirs, wherein the reservoirs comprise materials that are introduced into the first and/or second chambers; and
a plurality of means for providing the material from the reservoirs into the first and/or second chambers.

44. The apparatus of claim 38, wherein the first layer comprises a charged polymer, organic polycation, non-polymeric cation, or an inorganic material.

45. The apparatus of claim 38, wherein the first layer comprises polylysine.

46. The apparatus of claim 38, wherein the first layer comprises one or more charged species selected from the group consisting of histones, protamines, polyarginine, polyomithine, DEAE dextran, polybrene, and polyethylenimine.

47. The apparatus of claim 38, wherein the first layer is electrostatically bound to the sensing surface.

48. The apparatus of claim 38, wherein the interaction between the first layer and the double-stranded nucleic acid generates an associated electrical response in the measurement circuit, a magnitude of the electrical response being correlated with a degree of interaction.

49. The apparatus of claim 38, wherein the interaction between the first layer and the double-stranded nucleic acid alters the electronic characteristics at the sensing surface, the alteration being indicative of the interaction.

50. The apparatus of claim 38, wherein the interaction between the first layer and the double-stranded nucleic acid alters a capacitance within the measurement circuit, the alteration being indicative of the interaction.

51. The apparatus of claim 49, wherein the electronic characteristic is at least one of capacitance, conductance, impedance, resistance, current, voltage, and electric field intensity.

52. The apparatus of claim 38, wherein the property of the interaction between the first layer and the double-stranded nucleic acid introduced into the first chamber after the at least one cycle of DNA amplification that is measured is mass.

53. The apparatus of claim 38, wherein the property of the interaction between the first layer and the double-stranded nucleic acid introduced into the first chamber after the at least one cycle of DNA amplification that is measured is thickness.

54. The apparatus of claim 38, wherein the measurement circuit comprises:
a charge-sensitive region underlying the sensing surface;
an electrolyte solution disposed on the sensing surface; and
a semiconductor region at least partially surrounding the charge-sensitive region, wherein the sensing surface, charge-sensitive region, semiconductor region, and electrolyte solution form at least one capacitor.

55. The apparatus of claim 54, wherein the charge-sensitive region and at least a portion of the semiconductor region form a silicon sensor on a planar substrate.

56. An apparatus for detecting and optionally quantifying a double-stranded nucleic acid comprising:
a first chamber containing a sensing surface with an associated first charge;
an X layer comprising a charged species, wherein the X layer is bound to the X−1 layer, and wherein the X layer has an associated second charge opposite to the first charge so that the X layer and sensing surface together create a second charge or neutral charge on a net basis;
a second chamber for performing DNA amplification reactions;
a means for removing a sample of double-stranded nucleic acid from the second chamber after a Y cycle of DNA amplification and introducing the sample into the first chamber; and
a measurement circuit operatively connected to the first chamber, for measuring a property of an interaction between the X layer and the double-stranded nucleic acid introduced into the first chamber after the Y cycle of DNA amplification.

57. The apparatus of claim 56, further comprising:
a reservoir, wherein the reservoir comprises material for forming the X layer; and
a means for providing the material for forming the X layer into the first chamber.

58. The apparatus of claim 56, further comprising:
a plurality of reservoirs, wherein the reservoirs comprise materials that are introduced into the first and/or second chambers; and
a plurality of means for providing the material from the reservoirs into the first and/or second chambers.

59. The apparatus of claim 56, wherein the X−1 layer is the sensing surface.

60. The apparatus of claim 56, wherein the second charge substantially neutralizes the first charge.

61. The apparatus of claim 56, wherein the first charge is a negative charge and wherein the second charge is positive.

62. The apparatus of claim 56, wherein the first charge is a positive charge and wherein the second charge is negative.

63. The apparatus of claim 56, wherein the X layer is a charged polymer, organic polycation, non-polymeric cation, or an inorganic material.

64. The apparatus of claim 56, wherein the X layer comprises polylysine.

65. The apparatus of claim 56, wherein the X layer comprises one or more charged species selected from the group consisting of histones, protamines, polyarginine, polyornithine, DEAE dextran, polybrene, and polyethylenimine.

66. The apparatus of claim 56, wherein the X layer is electrostatically bound to the sensing surface.

67. The apparatus of claim 56, wherein the interaction between the X layer and the double-stranded nucleic acid generates an associated electrical response in the measurement circuit, a magnitude of the electrical response being correlated with a degree of interaction.

68. The apparatus of claim 56, wherein the interaction between the X layer and the double-stranded nucleic acid alters the electronic characteristics at the sensing surface, the alteration being indicative of the interaction.

69. The apparatus of claim 56, wherein the interaction between the X layer and the double-stranded nucleic acid alters a capacitance within the measurement circuit, the alteration being indicative of the interaction.

70. The apparatus of claim 56, wherein the electronic characteristic is at least one of capacitance, conductance, impedance, resistance, current, voltage and electric field intensity.

71. The apparatus of claim 56, wherein the property of the interaction between the X layer and the double-stranded nucleic acid introduced into the first chamber after the at least one cycle of DNA amplification that is measured is mass.

72. The apparatus of claim 56, wherein the property of the interaction between the X layer and the double-stranded nucleic acid introduced into the first chamber after the at least one cycle of DNA amplification that is measured is thickness.

73. The apparatus of claim 56, wherein the measurement circuit comprises:
a charge-sensitive region underlying the sensing surface;
an electrolyte solution disposed on the sensing surface; and
a semiconductor region at least partially surrounding the charge-sensitive region, wherein the sensing surface, charge-sensitive region, semiconductor region, and electrolyte solution form at least one capacitor.

74. The apparatus of claim 73, wherein the charge-sensitive region and at least a portion of the semiconductor region form a silicon sensor on a planar substrate.

75. The apparatus of claim 58, comprising a plurality of chambers for performing DNA amplification reactions.

76. The apparatus of claim 57, comprising a control means for controlling the introduction timing of the double-stranded nucleic acid and the X layer material into the chamber with the sensing surface.

77. The apparatus of claim 58, comprising a means for introducing a sample that may contain a nucleic acid of interest into the chamber for performing DNA amplification.

78. A kit comprising:
the apparatus of claim 56;
X layer material; and
a double-stranded nucleic acid sample.

79. The kit of claim 78, further comprising at least one of the following: amplification reagents, cleaning reagents, charged polymers, and pre-selected primers.


SEQUENCE LISTING (2 sequences)

SEQ ID NO: 1 - DNA, 20 bp, Artificial Sequence
  Forward primer
atcaagcagccatgcaaatg

SEQ ID NO: 2 - DNA, 21 bp, Artificial Sequence
  Reverse primer
cctttggtccttgtcttatgt